(19) United States
(12) Patent Application Publication
(10) Pub. No.: US 2009/0198243 A1
(43) Pub. Date: Aug. 6, 2009

(54) DEVICE AND METHOD FOR STABILIZING A DAMAGED BONE WITH A BONE CEMENT MIXTURE

(76) Inventor: Jeffry S. Melsheimer, Springville, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/366,705

(22) Filed: Feb. 6, 2009

Related U.S. Application Data
(60) Provisional application No. 61/026,557, filed on Feb. 6, 2008.

(51) Int. Cl. A61B 17/58

(52) U.S. Cl. 606/93; 606/92

(57) ABSTRACT

In at least one embodiment, a device for introducing a bone cement mixture into a damaged bone of a patient is provided. The device comprises a needle including a proximal portion and a distal portion extending therefrom. An aperture is formed through the distal portion and the distal portion is configured for piercing into the damaged bone. A lumen is formed through the proximal and distal portions and is for advancing the bone cement mixture to the aperture to introduce the bone cement mixture into the damaged bone. The proximal and distal portions are configured for detaching the distal portion from the proximal portion to retain the distal portion within the damaged bone.

### REFERENCE TO RELATED APPLICATIONS

This application claims the benefit of U.S. Provisional Patent Application No. 61/026,557, filed Feb. 6, 2008, entitled DEVICE AND METHOD FOR STABILIZING A DAMAGED BONE WITH A BONE CEMENT MIXTURE, which is hereby incorporated herein in its entirety.

### BACKGROUND

1. Field of the Invention

The present disclosure relates to a device, a kit and a method for introducing a bone cement mixture into a damaged bone of a patient.

2. Background

There is a clinical need to fill and stabilize damaged bones of patients, such as for example, filling defects and collapsed vertebra of patients suffering from severe back pain caused by osteoporosis, metastatic tumors or back injuries. Currently, these defects are repaired using multi-component bone cements that are mixed in open containers, transferred to a device and injected from the device into the damaged bone where the mixture chemically reacts or cures to form a solid structure.

The most widely used bone cements are based on polymethylmethacrylate (PMMA) and hydroxyapatite. These materials have relatively good strength characteristics, but have a number of drawbacks. These cements are a two-part chemically reactive system and have approximately 5-10 minutes of working time once the components are mixed together to form a bone cement mixture. The bone cement mixture is, for example, then injected into a collapsed vertebra of a patient, typically with a syringe in fluid communication with a large bore needle that has been inserted into the vertebra. Once the vertebra is filled with the bone cement mixture, the large bore needle is retracted from the vertebra. However, several problems can occur with this procedure.

One problem is that the bone cement may form a slug or slugs, which while anchored to the vertebra, continues through the tissue and protrudes out the back of the patient. For example, a large bore needle may continue to advance the highly viscous bone cement mixture through its open distal end while the needle is being retracted from the vertebra, leaving remnants of the bone cement mixture attached to the vertebra. These remnants cure, becoming a solid structure or structures otherwise known as a slug or slugs. Some of the slugs may be long and protrude from the body, or in other cases, they may be short making them difficult to access. In either case, the slugs are problematic for the patient and often, they must be removed. Removal of the slugs, however, can sometimes be difficult to do.

Another problem is that the bone cement mixture cures prior to the large bore needle being fully retracted from the vertebra. In this scenario, the needle may become affixed to the vertebra. In one example, the solidified bone cement mixture disposed in the needle is bonded to the solidified bone cement mixture that fills the vertebra. In another example, the bone cement mixture leaks back through the annular space between an access hole formed in the vertebra and the cannula of the needle. Because of the high tensile strength of the solidified bone cement mixture, the bone cement bond may be difficult to break or “snap apart” by pulling and/or twisting the needle.

Yet another problem is that the bone may become weakened by the access hole for the needle, which has been bored into the bone. For example, the vertebra may have an access hole bored through its pedicle which has become weakened by the boring out of the underlying bone structure. The weakened pedicle may be problematic for the patient.

### BRIEF SUMMARY

In at least one embodiment, a device for introducing a bone cement mixture into a damaged bone of a patient is provided. The device comprises a needle including a proximal portion and a distal portion extending therefrom. An aperture is formed through the distal portion. The distal portion is configured for piercing into the damaged bone. A lumen is formed through the proximal and distal portions and is for advancing the bone cement mixture to the aperture, introducing the bone cement mixture into the damaged bone. The proximal and distal portions are configured for detaching the distal portion from the proximal portion to retain the distal portion within the damaged bone.

In one aspect, the proximal and distal portions are configured to detach the distal portion from the proximal portion such that the distal portion does not protrude from the damaged bone.

In another aspect, the distal portion is retained permanently within the damaged bone, such as for example as an implant within a pedicle of a vertebra, and reinforces the damaged bone.

In yet another aspect, the distal portion is made from a biocompatible material.

In at least one other embodiment, a bone cement substitute kit for introducing a bone cement mixture into the damaged bone of a patient is provided. The kit comprises a first bone cement component and a second bone cement component that form the bone cement mixture. An injection device, including an outlet, is for containing the bone cement mixture. In one aspect, the injection device is also be used for mixing the bone cement mixture. The injection device is configured to dispense the bone cement mixture by advancing the bone cement mixture through the outlet. A needle is in fluid communication with the outlet. The needle includes a proximal portion and a distal portion extending therefrom and has an aperture formed through the distal portion. The distal portion is configured for piercing into the damaged bone. A lumen is formed through the proximal and distal portions and is for advancing the bone cement mixture to the aperture, introducing the bone cement mixture into the damaged bone. The proximal and distal portions are configured for detaching the distal portion from the proximal portion to retain the distal portion within the damaged bone.

In at least one other embodiment, a method for introducing a bone cement mixture into a damaged bone of a patient is provided. The method comprises piercing the damaged bone with a distal portion of a needle. The needle includes a proximal portion and a distal portion extending therefrom. The proximal and distal portions have a lumen formed therethrough. The bone cement mixture is advanced through the lumen to an aperture formed through the distal portion, introducing the bone cement mixture to the damaged bone. The distal portion is detached from the proximal portion to retain the distal portion within the damaged bone.

Further objects, features and advantages will become apparent from consideration of the following description and the appended claims when taken in connection with the accompanying drawings.

### BRIEF DESCRIPTION OF THE DRAWINGS

FIG. 1 is a perspective view of a collapsed vertebra.

FIG. 2a is a partial side view of a device for stabilizing a collapsed vertebra.

FIG. 2b is a top view of the FIG. 2a device inserted in the collapsed vertebra.

FIG. 3 is an enlarged view of FIG. 2a.

FIG. 4a is a partial side view of a device for stabilizing a collapsed vertebra.

FIG. 4b is a partial side view of a device for stabilizing a collapsed vertebra.

FIG. 5 is a side view of a stabilized collapsed vertebra.

FIG. 6 is a perspective view of a device for stabilizing a collapsed vertebra.

FIG. 7a is an exploded view of the device depicted in FIG. 6.

FIG. 7b is a perspective view of an alternate embodiment of the FIG. 7a obturator.

FIG. 8a is an enlarged partial cross-section of an exploded view of the device for stabilizing a collapsed vertebra.

FIG. 8b is an enlarged partial cross-section of an exploded view of the device for stabilizing a collapsed vertebra.

FIG. 8c is an enlarged partial cross-section view of the device for stabilizing a collapsed vertebra.

FIG. 8d is an enlarged partial cross-section of an exploded view of the device for stabilizing a collapsed vertebra.

FIG. 8e is an enlarged partial side view of the device for stabilizing a collapsed vertebra.

FIG. 9 is a side view of a bone cement substitute kit.

FIG. 10 is an exploded view of a bone substitute kit.

FIG. 11 is a flow chart for a method for introducing a bone cement mixture into a damaged bone of a patient.

FIG. 12a is a side view of an alternate embodiment of the device for stabilizing a collapsed vertebra.

FIG. 12b is a side view of an alternate embodiment of the device for stabilizing a collapsed vertebra.

FIG. 12c is a side view of an alternate embodiment of the device for stabilizing a collapsed vertebra.

FIG. 12d is a side view of an alternate embodiment of the device for stabilizing a collapsed vertebra.

### DETAILED DESCRIPTION

Detailed embodiments are disclosed herein. It is understood however, that the disclosed embodiments are merely exemplary of the invention and may be embodied in various and alternative forms. The figures are not necessarily to scale; some figures may be configured to show the details of a particular component.

Therefore, specific structural and functional details disclosed herein are not to be interpreted as limiting but merely as a representative basis with the claims and for teaching one skilled in the art to practice the present invention.

Examples of the present invention seek to overcome some of the concerns associated with stabilizing the damaged bone of a patient with a bone cement mixture while minimizing the likelihood of leaving a slug or slugs of the bone cement mixture attached to the bone and/or facilitating removal of a needle that has become inadvertently affixed to the bone during such a procedure and/or minimizing weakening of the bone by an access hole for the needle used to dispense the bone cement.

Disclosed is a device, a kit and a method for introducing a bone cement mixture into a damaged bone of a patient. The device, which is utilized in both the kit and the method, includes a needle that is used for introducing the bone cement mixture into the damaged bone. The needle includes a detachably connected distal portion that pierces into the damaged bone, forming a substantially cylindrical wall through a portion of the bone to define a bone opening or access hole. The bone cement mixture is advanced through an aperture, which is formed in the distal portion, introducing the bone cement mixture into the damaged bone. The distal portion may then be detached from the remaining portion of the needle, retaining the distal portion within the damaged bone.

In one example, the distal portion contains a slug of bone cement. By retaining the distal portion within the damaged bone such that the distal portion does not protrude from the bone, the slug is contained within the bone without protruding therefrom. In another example, the bone cement cures and/or solidifies prior to the needle being retracted. The needle is retracted by detaching the distal portion, which is affixed to the bone by the bone cement, from the remaining portion of the needle. The remaining portion of the needle is removed from the patient's body, thereby leaving the distal portion within and affixed to the bone. In yet another example, a portion of the damaged bone is weakened by the access hole for the needle. The distal portion of the needle is retained within the damaged bone, preferably within the access hole, providing structure to the weakened portion of the bone. The distal portion may also include a slug of bone cement, which may further enhance structural support to the bone.

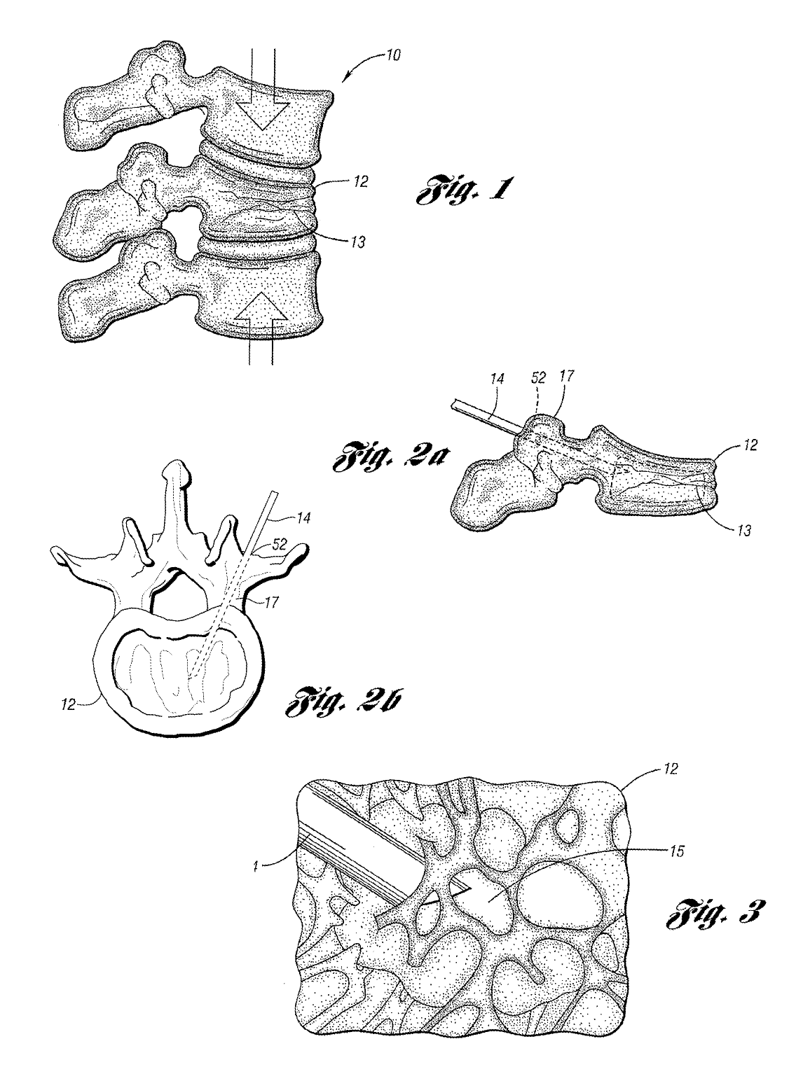

Referring now to the drawings, FIG. 1 illustrates a vertebrae 10 which includes a collapsed vertebra 12 with a compression fracture 13. The vertebra 10 may be, for example, in the thoracic or lower spine of the patient. In the compression fracture 13 of the vertebra 12, the bone tissue of the vertebral body collapses. This condition is commonly caused by osteoporosis and less often by tumor, or trauma to the back.

Referring now to FIGS. 2a-b and 3, at least one embodiment is provided for stabilizing collapsed vertebra 12 by either vertebroplasty or kyphoplasty, both of which are medical procedures for restoring structural integrity to the collapsed vertebra 12. These procedures stabilize the collapsed vertebra 12 by filling in open spaces 15 within the vertebra 12 to provide a more continuous and solid form. Kyphoplasty may further stabilize the vertebra 12 by restoring vertebral spacing which alleviates nerve pinching from the vertebra 12. It should be noted that the disclosed device and method applies to both of these medical procedures and other procedures for stabilizing and/or repairing the damaged bone of patients despite many of the various embodiments discussed herein as describing using vertebroplasty.

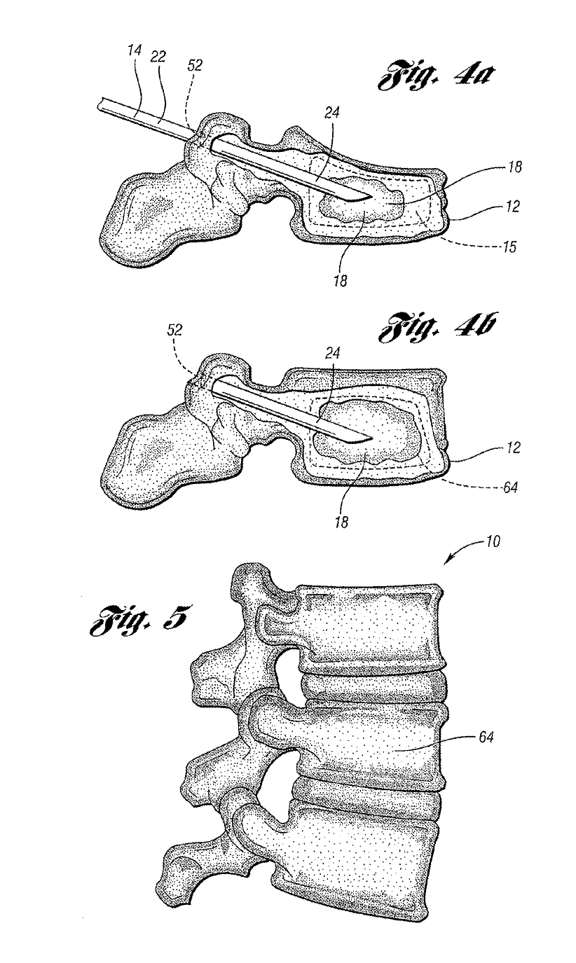

Vertebroplasty requires that the patient remain in a prone position throughout the entire procedure. Typically, this procedure is performed using X-ray to enhance visualization. It is performed under local anesthesia and/or a light sedative. A small nick is then made in the skin near the spine and a needle 14 is inserted percutaneously. As illustrated in FIG. 3, the needle 14 may be inserted into the interior open spaces 15 of the vertebra 12, for example via or through the left or right pedicle 17 of the vertebra 12.

Referring to FIGS. 4a-5, the bone cement mixture 18 may be dispensed from a syringe or other injection device through the needle 14 and into the vertebra 12 to form a solid structure 64 that supports the collapsed vertebra 12. The bone cement mixture 18 forms a solid structure 64 by chemically reacting or curing to become a solid. The stabilizing structure 64 may be formed within and/or about the collapsed vertebra 12 and may help restore vertebral spacing and alleviate nerve pinching by supporting the collapsed vertebra 12 generally in at least a compressive mode. Preferably, the structure 64 substantially fills in the open space 15 of the collapsed vertebra 12 providing a more dense and continuous vertebra 12 which enhances the mobility of, and alleviates pain in the patient.

Referring to FIGS. 4a-8e, at least one embodiment of a device 20 for introducing a bone cement mixture 18 into a damaged bone 12 of a patient is provided. The device 20 comprises a needle 14, a connector 29 and a grip 70. The needle 14 includes a proximal portion 22 and a distal portion 24 extending from the proximal portion 22. The proximal portion 22, which may be in the form of a cannula, may be straight or curved, or may flex between being straight and curved. The needle 14 and/or the proximal portion 22 and/or the distal portion 24 may be made of stainless steel, titanium, a superelastic metal such as a superelastic nickel-titanium alloy, Nitinol, or any other suitable metallic or non-metallic material known to those skilled in the art. In one example, the proximal portion 22 and the distal portion 24 are each made from different materials. For instance, the proximal portion 22 may be made of stainless steel while the distal portion 24 may be made from one of polymethylmethacrylate (PMMA), Nitinol, and Hydroxyapatite. In another example, the distal portion 24 is made from a suitable biocompatible material.

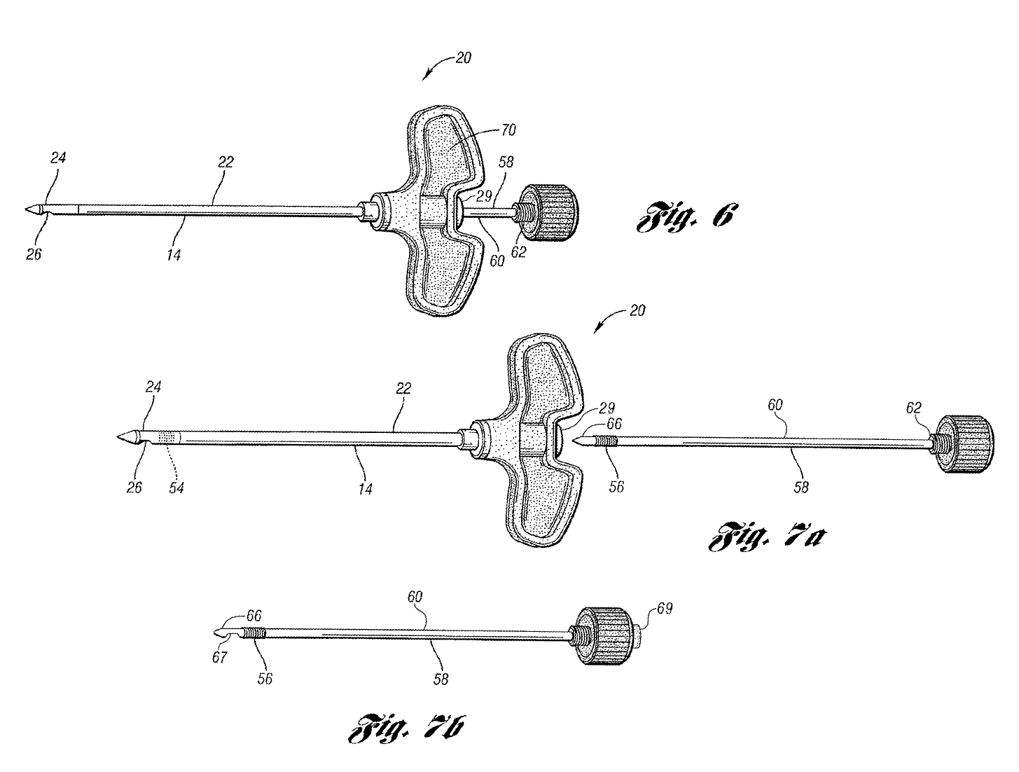

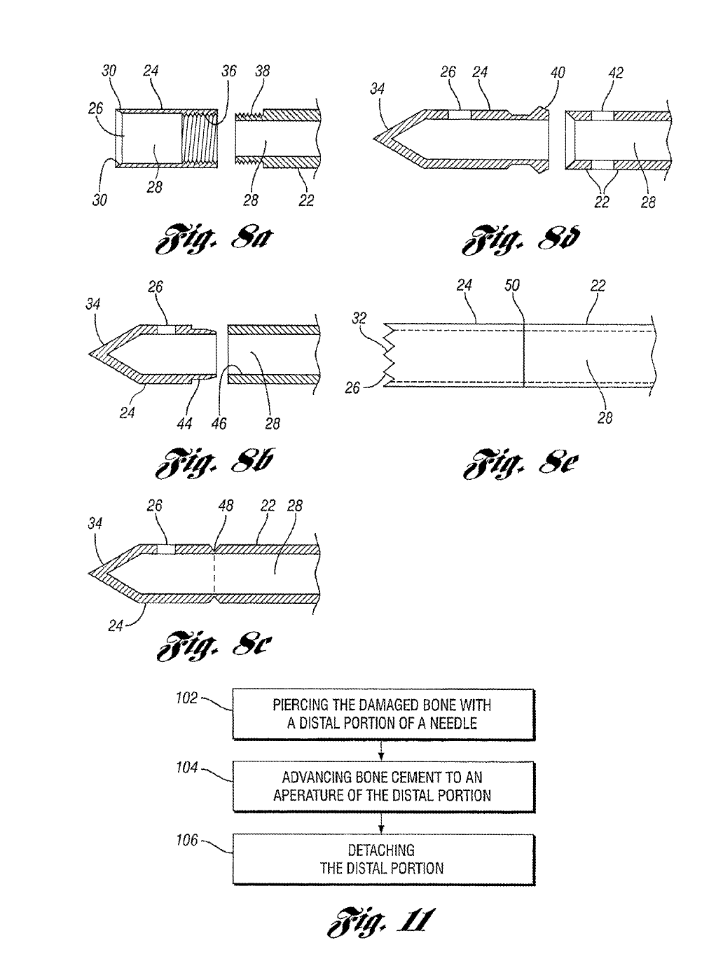

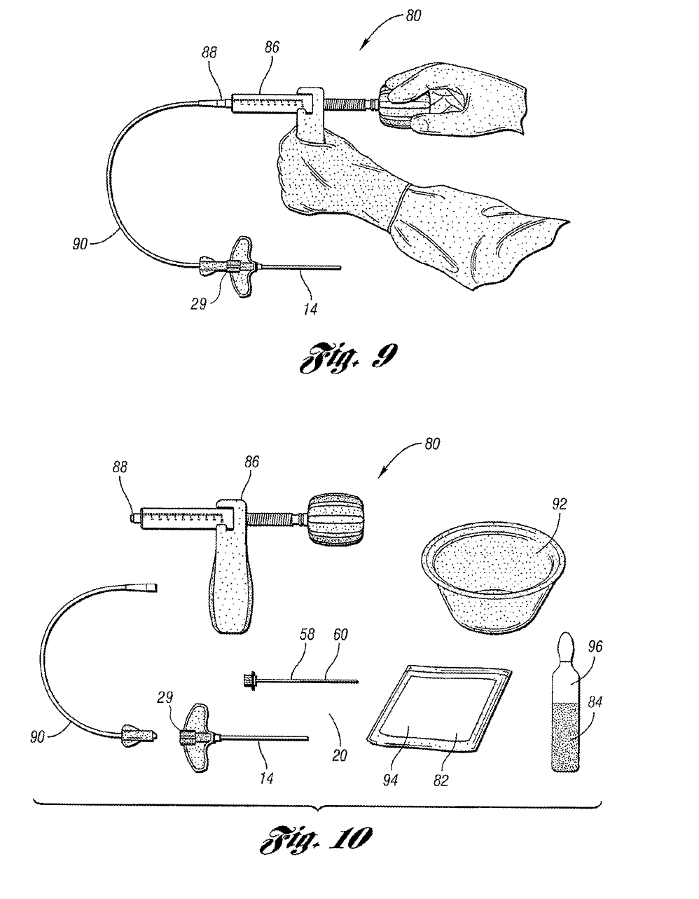

The distal portion 24 has an aperture 26 formed through the distal portion 24. The aperture 26 may be an opening formed at an end of the distal portion 24 (depicted in FIGS. 8a and 8e) or an opening formed in a side of the distal portion 24 (depicted in FIGS. 6-7 and 8b-8d). The aperture 26 is for introducing the bone cement mixture 18 into the damaged bone 12.

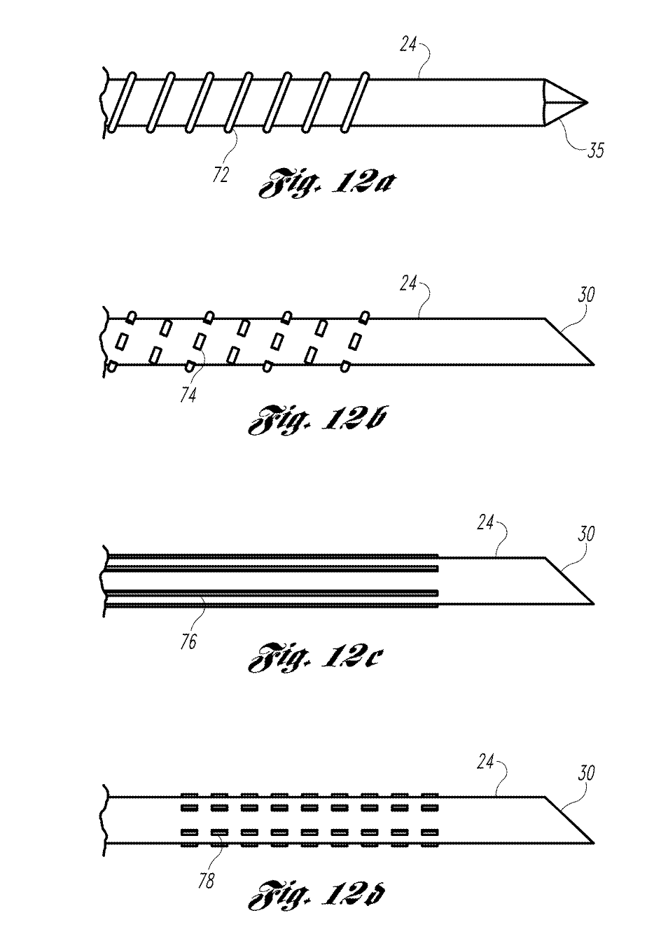

The distal portion 24 is configured for making an access hole 52 into and/or through the damaged bone 12, hereinafter referred to as piercing into the damaged bone 12. For example, the distal portion 24 may include a beveled cutting edge 30, or saw-toothed cutting edge 32, a pointed end 34 and/or a trocar tip 35 for piercing into the damaged bone 12 to make access hole 52. Further in this regard, distal portion 24 can be sized in length and diameter to fit various vertebra in different sized individuals. Accordingly, the length and diameter of distal portion 24 may vary for different applications. In this regard, it will be understood that the diameter, length and aspect ratio of needle 14 and distal portion 24 are not drawn to scale. In particular, FIGS. 8a-8e and 12a-12d are illustrative only, as they are not drawn to scale or with regard to any aspect ratio.

A lumen 28 is formed through the proximal and distal portions 22 and 24 and is in fluid communication with the aperture 26. The bone cement mixture 18 is advanced to the aperture 26 via the lumen 28 to introduce the bone cement mixture 18 to the damaged bone 12. The damaged bone 12 is then filled with the bone cement mixture 18.

The proximal and distal portions 22 and 24 are configured for detaching the distal portion 24 from the proximal portion 22 such that the distal portion 24 may be retained within the damaged bone 12. In at least one embodiment, the distal portion 24 is detachably connected to the proximal portion 22. In one example and as illustrated in FIG. 8a, the distal portion 24 is connected to the proximal portion 22 by a rotational coupling or engaging threads 36 on the distal portion with threads 38 on the proximal portion 22. The distal portion 24 may be detached from the proximal portion 22 (see FIG. 4b) by “unscrewing” and/or rotating the portions 22 and 24 so as to disengage the threads 36 and 38 or other suitable engaging features.

In another example, the distal portion 24 is attached to the proximal portion 22 by a snap fit connection. For instance and as illustrated in FIG. 8d, the distal portion 24 may have positive features 40 which may be snapped into or received by negative feature 42 formed on the proximal portion 22. The distal portion 24 may be detached from the proximal portion by “unsnapping” or disengaging the features 40 and 42. Snap fit connections and features are well known in the art and any suitable configuration may be used.

In yet another example, the distal portion 24 is attached to the proximal portion 22 by a slip fit connection or an interference fit connection. For instance and as illustrated in FIG. 8b, the distal portion 24 may have an extending portion 44 or portions located at its proximal end. The extending portion 44 may fit tightly within a distal wall 46 or walls located at the distal end of the proximal portion 22. The tight fit of the extending portion 44 and the distal wall 46 is such that the connection between the distal portion 24 and the proximal portion 22 may become detached by retracting the proximal portion 22 from the distal portion 24 with a suitable amount of force. Slip fit and interference fit connections are also well known in the art and any suitable configuration may be used.

In another example and as illustrated in FIG. 8c, the distal portion 24 is connected to the proximal portion 22 by a breakable stress riser 48. The breakable stress riser 48 may be for example, a “V” groove formed in the wall of the needle 14 which when flexed, concentrates a flexing force such that the distal portion 24 breaks away from the proximal portion 22. Other suitable breakable stress concentrators and pre-weakening shaped grooves may also be used to detachably connect the portions 22 and 24.

In still yet another example as illustrated in FIG. 8e, the distal portion 24 is attached to the proximal portion 22 by a frangible seam 50. The frangible seam 50, much like the breakable stress riser 48, is a weakening in the wall of the needle 14 but without any substantial reduction in the wall thickness of the needle 14. For instance, the frangible seam 50 may be formed by a two shot injection molding process used to make the needle 14. The two shot injection molding process may form a knit line, defining the frangible seam 50, where the two shots meet and partially bond together. Alternatively, the frangible seam 50 may be formed by an ultrasonic or a hot plate welding bond formed between the portions 22 and 24. Moreover, two different materials, which preferably form an incomplete or partial bond to one another, may be used for either the two shot injection molding or welding process, which may further weaken the frangible seam 50. Other suitable processes for forming a frangible seam may be used.

As illustrated in FIG. 4b, the distal portion 24 may be permanently retained within the damaged bone 12 to reinforce at least a portion of the damaged bone. For example, the distal portion 24 may be retained within vertebra 12 to reinforce the pedicle 17, which has an access opening 52 formed therethrough by the needle 14. In this example, the distal portion 24 may act as a biocompatible implant within vertebra 12. Furthermore, the distal portion 24 may be retained in the damaged bone 12 so as to not protrude from the damaged bone 12.

In one example, the distal portion 24 includes a radio pacifier, which is detectable by fluoroscopic visualization. The radio pacifier could be a biocompatible material that is substantially opaque to X-rays such as tungsten, gold or platinum. In other embodiments, distal portion 24 could include a number of small dimples in its surface that appear different under ultrasonic visualization, which can be used as an alternative to x-ray visualization methods such as fluoroscopy to determine whether distal portion 24 resides completely within the vertebra. This may allow an interventionalist to monitor the position of the needle 14, specifically the distal portion 24, within the patient's body during the medical procedure.

The device 20 may further comprise an obturator 58. The obturator 58 includes a shaft 60, a threaded portion 62 and a tip portion 66. The shaft 60 of the obturator 58 fits within the lumen 28 of the needle 14. The shaft 60 may be advanced through the lumen 28 towards the distal portion 24 so as to obstruct the aperture 26 such that when the distal portion 24 pierces into the damaged bone 12, the lumen 28 remains substantially free of fragments of the bone 12 or bone chips. Moreover, the obturator 58 may reinforce the needle 14 when the shaft 60 is disposed through the lumen 28, providing structural integrity for inserting the needle 14 into the damaged bone 12. In various embodiments, tip portion 66 may include cutting surfaces to aid distal portion 24 piercing into the damaged bone 12 to make access hole 52. In other embodiments, tip portion 66 may reinforce and support the distal portion 24 during insertion. In one embodiment, threaded portion 62 engages connector 29 to hold obturator 58 within needle 14. After insertion, obturator 58 can be removed from needle 14 by unthreading threaded portion 62 from connector 29.

In one embodiment as illustrated in FIG. 7a, obturator 58 may be secured to distal portion 24 by threads 54 on distal portion 24 threadingly engaged with external threads 56 on shaft 60 to further secure distal portion 24 during insertion of needle 14 into damaged bone 12. Distal portion 24 may then be detached from the obturator 58 by “unscrewing” and retracting shaft 60 through lumen 28, disengaging threads 56 from threads 54. In embodiments utilizing both threaded portion 62 engaging connector 29 and threads 56 engaging threads 54, the thread pitch for threaded portion 62 and threads 56 can be the same.

In another embodiment, the obturator 58 cooperates with the distal and proximal portions 22 and 24 to detachably connect the distal and proximal portions 22 and 24 together. For example, the distal portion 24 may have threads 54 which engage threads 56 formed on the shaft 60 so as to detachably connect the distal portion 24 to the proximal portion 22 when the shaft 60 is disposed through the lumen 28. In this embodiment, as illustrated in FIG. 7b, obturator 58 includes a hollow lumen connecting aperture 67 and connector 69 permitting obturator 58 to remain connected to the distal and proximal portions 22 and 24 when the bone cement mixture is dispensed into the vertebra. In this embodiment, the bone cement mixture can be dispensed through apertures 26 and 67 via connector 69. The distal portion 24 may then be detached from the proximal portion 22 by “unscrewing” and retracting the shaft 60 through the lumen 28, disengaging the threads 56 of the shaft 60 from the threads 54 of the distal portion 24.

Referring to FIGS. 9 and 10, at least one embodiment of a bone cement substitute kit for introducing a bone cement mixture into a damaged bone of a patient is provided. The kit 80 includes the device 20 as discussed in the foregoing paragraphs as well as a first bone cement component 82 and a second bone cement component 84. In one example, the first bone cement component 82 is contained in an envelope 94 and the second bone cement component is contained in a glass ampoule 96. The first and second bone cement components 82 and 84 may be removed from their respective containers 94 and 96 and mixed together within, for example, a mixing container 92 to form the bone cement mixture 18. In one example, the first bone cement component comprises methylmethacrylate, sodium phosphate, or a mixture thereof and the second bone cement component comprises polymethylmethacrylate, monocalcium phosphate, tricalcium phosphate, calcium carbonate or a mixture thereof. The first bone cement component may also contain a radio pacifier or radiopaque material such as derivatives of tungsten, barium, bismuth, etc.

An injection device 86 is for containing the bone cement mixture 18 and includes an outlet 88. In one example, the injection device 86 is a high pressure syringe capable of dispensing highly viscous bone cement mixtures 18 having a viscosity substantially similar to “paste.” The injection device 86 is configured to dispense the bone cement mixture 18 from the injection device 86 by advancing the bone mixture through the outlet 88.

The kit 80 may further include a tubing 90. The tubing 90 may be coupled to the outlet 88 of the injection device 86 and the connector 29 on the needle 14, providing fluid communication between the injection device 86 and outlet 88 and the needle 14.

Referring to FIG. 11, a method for introducing a bone cement mixture into the damaged bone of a patient is provided. The method includes piercing into the damaged bone with a distal portion of a needle 102. The needle includes a proximal portion and a distal portion extending from the proximal portion. A lumen is formed through the proximal and distal portions.

Referring to FIGS. 12a-d, alternative embodiments of the distal portion 24 are shown including external splined elements. Specifically, FIG. 12a illustrates a helical spline 72 around the outer periphery of distal portion 24. FIG. 12a also depicts a trocar tip 35. FIG. 12b illustrates segmented helical splines 74 around the outer periphery of distal portion 24. FIG. 12c illustrates straight splines 76 axially oriented on the outer periphery of distal portion 24. And FIG. 12d illustrates segmented straight splines 78 axially oriented on the outer periphery of distal portion 24. Splines 72, 74, 76 and 78 may includes a beveled leading edge to aid in forming access hole 52 to match the modified profile of distal portion 24.

Splines 72 and 74 may create a profile in the bone when access hole 52 is formed that could permit distal portion 24 to be advanced into vertebra 10 using a rotational advancement similar to a threaded engagement. Such engagement may be advantageous for accurately positioning distal portion 24 at the proper depth in vertebra 10. Splines 76 and 78 may create a profile in the bone when access hole 52 is formed that could resist any axial twisting of distal portion 24. Use of any of splines 72, 74, 76 and 78 may aid in detaching distal portion 24 from proximal portion 22. In addition, any of splines 72, 74, 76 and 78 may aid in retaining distal portion 24 securely within vertebra 10 after detaching from proximal portion 22.

The bone cement mixture is advanced through the lumen to an aperture formed through the distal portion 104 to introduce the bone cement mixture to the damaged bone. The distal portion is detached from the proximal portion 106 to retain the distal portion within the damaged bone.

The method may further comprise mixing a first bone cement component together with a second bone cement component to form the bone cement mixture. The bone cement mixture may be contained in an injection device in fluid communication with the lumen. The bone cement mixture may be dispensed from the injection device.

As a person skilled in the art will readily appreciate, the above description is meant as an illustration of the implementation of the principles of the invention claimed below. This description is not intended to limit the scope or application of the claims and the disclosure is susceptible to modification, variation and change, without departing from the spirit of this invention, as defined in the following claims.

I claim:

1. A device for introducing a bone cement mixture into a damaged bone of a patient, the device comprising:
a needle including a proximal portion and a distal portion extending therefrom, the distal portion having an aperture formed through the distal portion and being configured for piercing into the damage bone, the proximal and distal portions having a lumen formed therethrough for advancing the bone cement mixture to the aperture, introducing the bone cement mixture into the damaged bone, and being configured for detaching the distal portion from the proximal portion to retain the distal portion within the damaged bone.

2. The device according to claim 1 wherein the distal portion is detachably connected to the proximal portion by at least one of engaging threads, snap fit, slip fit, breakable stress riser, frangible seam, interference fit and rotational coupling.

3. The device according to claim 1 wherein the distal portion further comprises an external spline.

4. The device according to claim 3 wherein the configuration of the external spline is selected from the group consisting of a helical spline, a segmented helical spline, a straight spline and a segmented straight spline.

5. The device according to claim 1 wherein the distal portion is made from one of polymethylmethacrylate (PMMA), Nitinol, titanium and hydroxyapatite.

6. The device according to claim 1 wherein the distal portion is retained permanently within the damaged bone and reinforces at least a portion of the damaged bone.

7. The device according to claim 1 wherein the distal portion includes a radio pacifier detectable by fluoroscopic visualization.

8. The device according to claim 1 wherein the proximal and distal portions are configured to detach the distal portion from the proximal portion such that the distal portion does not protrude from the damaged bone.

9. The device according to claim 1 wherein the distal portion has at least one of a beveled cutting edge, a saw toothed cutting edge and a pointed end for piercing into the damaged bone.

10. The device according to claim 1 wherein the aperture is one of an open end and a side aperture for introducing the bone cement mixture into the damaged bone.

11. The device according to claim 1 further comprising an obturator including a shaft, the obturator being configured to advance the shaft through the lumen towards the distal portion, reinforcing the needle and obstructing the aperture such that when the distal portion pierces into the damaged bone the lumen remains substantially free of bone chips.

12. The device according to claim 11 wherein the obturator cooperates with the distal and proximal portions to detachably connect the distal and proximal portions together when the shaft is disposed within the lumen.

13. The device according to claim 11 wherein the proximal portion has a proximal end, the needle including a hub disposed adjacent to the proximal end of the proximal portion and the obturator including a fastening member attached to the shaft, the fastening member being configured to selectively couple to the hub of the needle.

14. A bone cement substitute kit for introducing a bone cement mixture into the damaged bone of a patient, the kit comprising:
a first bone cement component and a second bone cement component, the first and second bone cement components for forming the bone cement mixture; an injection device for containing the bone cement mixture and including an outlet, the injection device being configured to dispense the bone cement mixture from the injection device by advancing the bone cement mixture through the outlet; and a needle in fluid communication with the outlet and including a proximal portion and a distal portion extending therefrom, the distal portion having an aperture formed through the distal portion and being configured for piercing into the damage bone, the proximal and distal portions having a lumen formed therethrough for advancing the bone cement mixture to the aperture, introducing the bone cement mixture into the damaged bone, and being configured for detaching the distal portion from the proximal portion to retain the distal portion within the damaged bone.

15. The kit according to claim 14 further comprising a tubing configured to couple to both the outlet of the injection device and the needle and to provide fluid communication between the outlet and the needle.

16. The kit according to claim 14 further comprising an obturator including a shaft, the obturator being configured to advance the shaft through the lumen towards the distal portion, reinforcing the needle and obstructing the aperture such that when the distal portion pierces into the damaged bone the lumen remains substantially free of bone chips.

17. The kit according to claim 14 wherein the distal portion is detachably connected to the proximal portion by at least one of engaging threads, snap fit, slip fit, breakable stress riser, frangible seam, interference fit and rotational coupling.

18. The kit according to claim 14 wherein the distal portion is retained permanently within the damaged bone and reinforces at least a portion of the damaged bone.

19. A method for introducing a bone cement mixture into a damaged bone of a patient, the method comprising:
piercing into the damaged bone with a distal portion of a needle, the needle including a proximal portion and the distal portion extending therefrom, the proximal and distal portions having a lumen formed therethrough;
advancing the bone cement mixture through the lumen to an aperture formed through the distal portion to introduce the bone cement mixture to the damaged bone; and
detaching the distal portion from the proximal portion to retain the distal portion within the damaged bone.

20. The method according to claim 19 where the distal portion is detachably connected to the proximal portion by at least one of engaging threads, snap fit, slip fit, breakable stress riser, frangible seam, interference fit and rotational coupling and the step of detaching includes at least one of rotating the needle, pulling the needle and flexing the needle to disconnect the distal portion from the proximal portion.

21. The method according to claim 19 further including mixing a first bone cement component together with a second bone cement component to form the bone cement mixture, containing the bone cement mixture in an injection device in fluid communication with the lumen and the step of advancing the bone cement mixture includes dispensing the bone cement mixture from the injection device.
